SEQUENCE LISTING (68 sequences)

SEQ ID NO: 1 - protein, 177 aa, Homo sapiens
MACCQTSFCGFPSCSTSGTCGSSCCQPSCCETSSCQPRCCETSCCQPSCCQTSFCGFPSFSTGGTCDSSCCQPSCCETSCCQPSCYQTSSCGTGCGIGGGIGYGQEGSSGAVSTRIRWCRPDCRVEGTCLPPCCVVSCTPPSCCQLHHAEASCCRPSYCGQSCCRPVCCCYCSEPTC

SEQ ID NO: 2 - protein, 167 aa, Homo sapiens
MTCCQTSFCGYPSCSTSGTCGSSCCQPSCCETSCCQPSCCQTSFCGFPSFSTSGTCSSSCCQPSCCETSCCQPSCCQTSSCGTGCGIGGGIGYGQEGSSGAVSTRIRWCRPDCRVEGTCLPPCCVVSCTPPTCCQLHHAEASCCRPSYCGQSCCRPVCCCYSCEPTC

SEQ ID NO: 3 - protein, 174 aa, Homo sapiens
MTCCQTSFCGYPSFSISGTCGSSCCQPSCCETSCCQPRSCQTSFCGFPSFSTSGTCSSSCCQPSCCETSCCQPSCCETSCCQPSCCQISSCGTGCGIGGGISYGQEGSSGAVSTRIRWCRPDSRVEGTYLPPCCVVSCTPPSCCQLHHAQASCCRPSYCGQSCCRPVCCCEPTC

SEQ ID NO: 4 - protein, 131 aa, Homo sapiens
MACCQTSFCGFRSFSTGGTCDSSCCQPSCCETSCCQPSCYQTSSCGTGCGIGGGIGYGQEGSSGAVSTRIRWCRPDCRVEGTCLPPCCVVSCTPPSCCQLHHAEASCCRPSYCGQSCCRPVCCCYCSEPTC

SEQ ID NO: 5 - protein, 85 aa, Homo sapiens
MACCQTSFCGFPSCSTSGAVSTRIRWCRPDCRVEGTCLPPCCVVSCTPPSCCQLHHAEASCCRPSYCGQSCCRPVCCCYCSEPTC

SEQ ID NO: 6 - protein, 177 aa, Homo sapiens [sequence omitted]

SEQ ID NO: 7 - protein, 141 aa, Homo sapiens
MTCCQTSFCGYPSCSTSGTCGSSCCQPSCCETSCCQPSCCETSCCQPSCCQTSSCGTGCGIGGGIGYGQEGSSGAVSTRIRWCRPDCRVEGTCLPPCCVVSCTPPTCCQLHHAEASCCRPSYCGQSCCRPVCCCYSCEPTC

SEQ ID NO: 8 - protein, 96 aa, Homo sapiens
PVTCQTTVCRPVTCVPRCTRPICEPCRRPVCCDPCSLQEGCCRPITCCPSSCTAVVCRPCCWATTCCQPVSVQSPCCRPPCGQPTPCSTTCRTSSC

SEQ ID NO: 9 - protein, 33 aa, Homo sapiens
TTCCQPVSVQSPCCRPPCGQPTPCSTTCRTSSC

SEQ ID NO: 10 - protein, 85 aa, Homo sapiens
VTCVPRCTRPICEPCRRPVCCDPCSLQEGCCRPITCCPSSCTAVVCRPCCWATTCCQPVSVQSPCCRPPCGQPTPCSTTCRTSSC

SEQ ID NO: 11 - protein, 128 aa, Homo sapiens
MTGSCCGSTLSSLSYGGGCCQPCCCRDPCCCRPVTCQTTVCRPVTCVPRCTRPICEPCRRPVCCDPCSLQEGCCRPITCCPSSCTAVVCRPCCWATTCCQPVSVQSPCCRPPCGQPTPCSTTCRTSSC

SEQ ID NO: 12 - protein, 98 aa, Homo sapiens
MYCCALRSCSVPTGPATTFCSFDKSCRCGVCLPSTCPHEISLLQPICCDTCPPPCCKPDTYVPTCWLLNNCHPTPGLSGINLTTYVQPGCESPCEPRC

SEQ ID NO: 13 - protein, 98 aa, Homo sapiens
MDCCASRSCSVPTGPATTICSSDKSCRCGVCLPSTCPHTVWLLEPICCDNCPPPCHIPQPCVPTCFLLNSCQPTPGLETLNLTTFTQPCCEPCLPRGC

SEQ ID NO: 14 - protein, 98 aa, Homo sapiens
MDCCASRGCSVPTGPATTICSSDKSCRCGVCLPSTCPHTVWLLEPTCCDNCPPPCHIPQPCVPTCFLLNSCQPTPGLETLNLTTFTQPCCEPCLPRGC

SEQ ID NO: 15 - protein, 136 aa, Homo sapiens
MVNSCCGSVCSDQGCGLENCCRPSCCQTTCCRTTCCRPSCCVSSCCRPQCCQSVCCQPTCCSPSCCQTTCCRTTCCRPSCCVSSCFRPQCCQSVCCQPTCCRPSCGQTTCCRTTCYRPSCCVSTCCRPTCSSGSCC

SEQ ID NO: 16 - protein, 98 aa, Homo sapiens
TCCRPSCCISSCCRPSCCISSCCKPSCCQTTCCRPSCCISSCYRPQCCQPSCCRPACCISSCCHPSCCVSSCRCPFSCPTTCCRTTCFHPICCGSSCC

SEQ ID NO: 17 - protein, 166 aa, Homo sapiens
MVNSCCGSVCSDQGCGLENCCRPSYCQTTCCRTTCCRPSCCVSSCCRPQCCQTTCCRTTCCHPSCCVSSCCRPQCCQSVCCQPTCCRPQCCQTTCCRTTCCRPSCCRPQCCQSVCCQPTCCCPSYCVSSCCRPQCCQTTCCRTTCCRPSCCVSRCYRPHCGQSLCC

SEQ ID NO: 18 - protein, 186 aa, Homo sapiens
MVSSCCGSVSSEQSCGLENCCCPSCCQTTCCRTTCCRPSCCKPQCCQSVCYQPTCCHPSCCISSCCRPYCCESSCCRPCCCRPSCCQTTCCRTTCCRTTCCCPSCCVSSCCRPQCCQSVCCQPTCCRPSYCISSCCHPSCCESSCCRPCCCVRPVCGRVSCHTTCYRPTCVISTCPRPLCCASSCC

SEQ ID NO: 19 - protein, 109 aa, Homo sapiens
SPLAAVPAAASPAAVAPAAVCPGAAGPSAASLCAASQPAAVPAAASPAAAAPLAVNPAAAARAAAAPAAACVQSVAESPATPLAIAQPVSFPPAPVPCAVPPLAAELLA

SEQ ID NO: 20 - protein, 210 aa, Homo sapiens
MVSSCCGSVCSDQGCGQVLCQETCCRPSCCQTTCCRTTCYRPSCCVSSCCRPQCCQSVCCQPTCCRPSCCETTCCHPRCCISSCCRPSCCMSSCCKPQCCQSVCCQPTCCHPSCCISSCCRPSCCVSRCCRPQCCQSVCCQPTCCRPSCCISSCCRPSCCESSCCRPCCCRPCCCLRPVCGRVSCHTTCYRPTCVISTCPRPLCCASSCC

SEQ ID NO: 21 - protein, 114 aa, Homo sapiens
SSCCKPQCCQSVCCQPTCCHPSCCISSCCRPSCCVSSCCKPQCCQSVCCQPTCCHPSCSISSCCRPSCCESSCCRPCCCVRPVCGRVSCHTTCYRPTCVISTCPRPVCCASSCC

SEQ ID NO: 22 - protein, 191 aa, Homo sapiens
CQETCCRPSCCETTCCRTTCCRPSCCVSSCCRPQCCQSVCCQPTCSRPSCCQTTCCRTTCYRPSCCVSSCCRPQCCQPVCCQPTCCRPSCCETTCCHPRCCISSCCRPSCCVSSCCKPQCCQSVCCQPNCCRPSCSISSCCRPSCCESSCCRPCCCVRPVCGRVSCHTTCYRPTCVISSCPRPLCCASSCC

SEQ ID NO: 23 - protein, 127 aa, Homo sapiens
MVNSCCGSVCSDQGCDQGLCQETCCRPSCCQTTCCCPSCVVSSCCRPSCSQTTCCQTTCCRPSCCRPVCCQTTCRPSCGVSSCCRPLCCQTTCRPSCGVSSCCRPLCCQTTCCRTTCCRPSCCGSSC

SEQ ID NO: 24 - protein, 201 aa, Homo sapiens
MVNSCCGSVCSDQGCGLENCCRPSCCQTTCCRTTCCRPSCCVSSCCRPQCCQSVCCQPTCCRPSCCQTTCCRTTCCRPSCCVSSCCRPQCCQSVCCQPTCCRPSCCQTTCCRTTCCRPSCCVSSCCRPQCCQSVCCQPTCCRPSCCISSSCCPSCCESSCCRPCCCLRPVCGRVSCHTTCYRPTCVISTCPRPLCCASSCC

SEQ ID NO: 25 - protein, 106 aa, Homo sapiens
MVNSCCGSVCSDQGCGLENCCRPSYCQTTCCRTTCCRPSCCRPSCCRPQCCQSVCCQPTCCCPSYCVSSCCRPQCCQTTRCRTTCCRPSCCVSRCYRPHCGQSLCC

SEQ ID NO: 26 - protein, 195 aa, Homo sapiens
MVNSCCGSVCSHQGCGQDLCQETCCRPSCCETTCCRTTYCRPSCCVSSCCRPQCCQSVCCQPTCCRPRCCISSCCRPSCCVSSCCKPQCCQSMCCQPTCCRPRCCISSCCRPSCCVSSCCRPQCCQSVCCQPTCCHPSCSISSCCRPSCCESSCCRPCCCLRPVCGGVSCHTTCYRPTCVISSCPRPLCCASSCC

SEQ ID NO: 27 - protein, 193 aa, Homo sapiens
QGCGLETCCRPSCCQTTCCRTTCCRPSCCVSSCCRPQCCQSVCCQPTCCRPSCCPSCCQTTCCRTTCCRPSCCVSSCCRPQCCQSVCCQPTCCRPSCSISSCCRPSCCVSRCCRSQCCQSVCCQPTCCRPSCCISSCCRPSCCESSCCRPCCCRPCCCLRPVCGRVSCHTTCYRPTCVISTCPRPLCCASSCC

SEQ ID NO: 28 - protein, 278 aa, Homo sapiens
MGCCGCSGGCGSSCGGCGSGCGGCGSGCGGCGSGCGGSGSSCCVPVCCCKPVCCRVPTCSCSSCGKGGCGSSGGSKGGCGSCGGCKGGCGSCGGSKGGCGSCGGSKGGCGSCGGSKGGCGSGCGGCGSSCCVPVCCCKPMCCCVPACSCSSCGKGGCGSCGCSKGACGSCGGSKGGCGSCGGCKGGCGSCGGSKGGCGSGCGGCGSGCGVPVCCCSCSSCGSCAGSKGGCGSSCSQCSCCKPCCCSSGCGSSCCQSSCCKPCCSQSSCCVPVCCQCKI

SEQ ID NO: 29 - protein, 177 aa, Homo sapiens
MGCCGCSRGCGSGCGGCGSSCGGCGSGCGGCGSGRGGCGSGCGGCSSSCGGCGSRCYVPVCCCKPVCSWVPACSCTSCGSCGGSKGGCGSCGGSKGGCGSCGGSKGGCGSCGCSQSSCCKPCCCSSGCGSSCCQSSCCKPCCCQSSCCVPVCCQSSCCKPCCCQSNCCVPVCCQCKI

SEQ ID NO: 30 - protein, 238 aa, Homo sapiens
MGCSGCSGGCGSSCGGCGSSCGGCGSGYGGCGSGCCVPVCCCKPVCCCVPACSCSSCGSCGGSKGVCGSCGGCKGGCGSCGGSKGGCGSSCCVPVCCSSSCGSCGGSKGVCGFRGGSKGGCGSCGCSQCSCYKPCCCSSGCGSSCCQSSCCKPSCSQSSCCKPCCSQSSCCKPCCCSSGCGSSCCQSSCCKPCCSQSSCCKPCCCSSGCGSSCCQSSCCKPCSSQSSCCVPICCQCKI

SEQ ID NO: 31 - protein, 288 aa, Homo sapiens
MGCCGCSGGCGSGCGGCGSGCGGCGSGCGGCGSGCGGCGSGCGGCGSSCCVPICCCKPVCCCVPACSCSSCGSCGGSKGGYGSCGGSKGGCVSCGGSKGGCGSCGGSKGGCGSCGGSKGGCGSCGGSKGGCVSCGGSKGGCGSCGGSKGGCVSCGGSKGGCGSCGGSKGGCGSCGGSKGGCGSCGGSKGGCGSCGCSQCSCCKPCCCSSGCGSSCCQSSCCKPCCSSSGCGSSCCQSSCCKPYCCQSSCCKPCCSSSGCGSSCCQSSCCNPCCSQSSCCVPVCCQCKI

SEQ ID NO: 32 - protein, 221 aa, Homo sapiens
MGCCGCSGGCGSGCGGLGSGCGGCGSGCGGRGSGCGGCGSGCGGCGSGCGGCGSGCGGCGGCGSGCCVPVCCCKPMCCCVPACSCSSCGKGGCGSCGGSKRGCVSCGVSKGACGSCGGSKGGCGSCGGSKGGCGSCGGSKGGCGSYGCSQCSCCKPCCCSSGCGSSCCQSSCCKPYCCQSSCCKPCSCFSGCGSSCCQSSCYKPCCCQSSCCVPVCCQCKI

SEQ ID NO: 33 - protein, 129 aa, Homo sapiens
MGCCGCSGGCGSGCGGCGSGCGGCGSSCCVPICCCKPVCCCVPACSCTSCGSCGGSKGCCGSCGGSKGGCGSCGGSKGGCGSCGCSQCSCCKPCYCSSGCGSSCCQSSCCKPCCSQASCCVPICCQCKI

SEQ ID NO: 34 - protein, 165 aa, Homo sapiens
MGCCGCSEGCGSGCGGCGSGCGGCGSGCGGCGSSCCVPVCCCKPVCCCVPACSCSSCGSCGGSKGGCGSCGGSKGGCGSCGGSKGGCGSCGCSQCSCYKPCCCSSGCGSSCCQSSCCKPCCCQSSCCKPCCCSSGCGSSCCQSSCCNPCCSQSSCCVPVCCQCKI

SEQ ID NO: 35 - protein, 187 aa, Homo sapiens
MGCCGCSGGCGSGCGGCGSGCGGCGSSCCVPICCCKPVCCCVPACSCSSCGSCGGSKGGRGSCGGSKGDCGSCGGSKGGCGSCGCSQCSCYKPCCCSSGCGSSCCQSSCCKPCCSQSSCCKPCSCSSGCGSSCCQSSCCKPCCSQSSCCKPCCCSSGCGSSCCQSSCCKPCCSQSSCCVPICCQCKI

SEQ ID NO: 36 - protein, 169 aa, Homo sapiens
MGCCGCSGGCGSSCGGCDSSCGSCGSGCRGCGPSCCAPVYCCKPVCCCVPACSCSSCGKRGCGSCGGSKGGCGSCGCSQCSCCKPCCCSSGCGSSCCQCSCCKPYCSQCSCCKPCCSSSGRGSSCCQSSCCKPCCSSSGCGSSCCQSSCCKPCCSQSRCCVPVCYQCKI

SEQ ID NO: 37 - protein, 202 aa, Homo sapiens
MGCCGCSGGCGSGCGGCGSGCGGCGSGCGGYGSGCGGCGSSCCVPVCCCKPVCCCVPACSCSSCGSCGGSKGDCGSCGGSKGGCGSCGGSKGGCGSCGGSKGGCGSCGGSKGGCGSCGGSKGGCGSCGGSKGGCGSCGCSQCNCCKPCCCSSGCGSCCQSSCCNPCCCQSSCCVPVCCQSSCCKPCCCQSSCCVPVCCQCKI

SEQ ID NO: 38 - protein, 156 aa, Homo sapiens
MGCCGCSGGCGSGCGGCGSGSGGCGSGCGGCGSSCCVPICCCKPVCCCVPACSCSSCGSCGGSKGGCGSCGSSKGGCGSCGCSQSNCCKPCCSSSGCGSFCCQSSCSKPCCCQSSCCQSSCCKPCCCQSSCCQSSCFKPCCCQSSCCVPVCCQCKI

SEQ ID NO: 39 - protein, 87 aa, Homo sapiens
MTRYFCCGSYFPGYPIYGTNFHGTFRATPLNCVVPLGSPLNYGCGCNGYSSLGYSFGGSNINNLGGCYGGSFYRPWGSGSGFGYSTY

SEQ ID NO: 40 - protein, 63 aa, Homo sapiens
MLCDNFPGAVFPGCYWGSYGYPLGYSVGCGYGSTYSPVGYGFGYGYNGCGAFGYRRYSPFALY

SEQ ID NO: 41 - protein, 174 aa, Homo sapiens [sequence omitted]

SEQ ID NO: 42 - protein, 159 aa, Homo sapiens
MTHCCSPCCQPTCCRTTCWQPTTVTTCSSTPCCQPSCCVSSCCQPCCHPTCCQNTCCRTTCCQPICVTSCCQPSCCSTPCCQPTCCGSSCGQSSSCAPVYCRRTCYHPTSVCLPGCLNQSCGSNCCQPCCRPACCETTCCRTTCFQPTCVYSCCQPSCC

SEQ ID NO: 43 - protein, 154 aa, Homo sapiens
MTHCCSPCCQPTCCRTTCCRTTCWKPTTVTTCSSTPCCQPSCCVSSCCQPCCRPTCCQNTCCQPTCVTSCCQPSCCSTPCCQPTCCGSSCDQSSSCAPVYCRRTCYYPTTVCLPGCLNQSCGSNCCQPCCRPACCETTCFQPTCVYSCCQPFCC

SEQ ID NO: 44 - protein, 111 aa, Homo sapiens
TTCCQPTCLTSCCQPSCCSTTCCQPICCGSSCCGQTSCGSSCGQSSSCAPVYCRRTCYYPTTVCLPGCLNQSCGSSCCQPCCRPACCETTCCRTTCFQPTCVSSCCQPSCC

SEQ ID NO: 45 - protein, 159 aa, Homo sapiens
MTHCCSPCCQPTCCRTTCWKPTTVTTCSSTPCCQPSCCVSSCCQPCCRPTCCQNTCCQPICVTSCCQPSCCSTPCCQPTCCGQTSCGSSCGQSSSCAPVYCRRTCYHPTTVCLPGCLNQSCGSSCCQPCCRPACCETTCCRTTCFQPTCVYSCCQPSCC

SEQ ID NO: 46 - protein, 154 aa, Homo sapiens
MTHCCSPCCQPTCCRTTCCRTTCWKPTTVTTCSSTPCCQPSCCVSSCCQPCCRPACCQNTCCRTTCCQPTCLSSCCGQTSCGSSCGQSSSCAPVYCRRTCYYPTTVCLPGCLNQSCGSSCCQPCCRPACCETTCCRTTCFQPTCVSSCCQPSCC

SEQ ID NO: 47 - protein, 282 aa, Homo sapiens
MAASTMSVCSSACSDSWQVDACPESCCEPHCCALSCCAPAPCLTLVCTPVSRVSSPCCQAACEPSPCQSGCTSSCTPSCCQQSSCQPACCTSSPCQQACCVPVCCKPVCCLPTCSKDSSSCCQQSSCQPTCCASSSSQQSCCVPVCCKPVCYVPTCSEDSSSCCQQSSCHPACCTSSPCQQACCVPVRCKPVCCKPICCVPVCSGASTSCCQQSSCQPACCTTSCCRPSSSVSLLCRPVCRPACCMPVSSCCAPASSCQASCCRPASCVSLLCRPACSRPAC

SEQ ID NO: 48 - protein, 255 aa, Homo sapiens
MAASTMSICSSACTNSWQVDDCPESCCELPCGTPSCCAPAPCLTLVCTPVSCVSSPCCQAACEPSACQSGCTSSCTPSCCQQSSCQPACCTSSPCQQACCVPVCCKPVCCVPVCCGASSCCQQSSCQPACCASSSCQQSCRVPVCCKAVCCVPTCSESSSSCCQQSSCQPACCTSSPCQQSCCVSVCCKPVCCKSICCVPVCSGASSPCCQQSSCQPACCTSSCCRPSSSVSLLCRPVCSRPASCSFSSGQKSSC

SEQ ID NO: 49 - protein, 221 aa, Homo sapiens
MATSTMSVCSSAYSDSWQVDACPESCCEPPCCATSCCAPAPCLTLVCTPVSCVSSPCCQAACEPSPCQSGCTSSCTPSCCQQSSCQPACCTSSPCQQACCVPVCCKPVCCVPVCCKPVCCKPICCVPVCSGASSSCCQQSSRQPACCTTSCCRPSSSVSLLCRPVCRSTCCVPIPSCCAPASTCQPSCCRPASCVSLLCRPTCSRLSSACCGLSSGQKSSC

SEQ ID NO: 50 - protein, 401 aa, Homo sapiens
MSVCSSDLSYSSRVCLPGSCDSCSDSWQVDDCPESCCEPPCCAPSCCAPAPCLSLVCTPVSRVSSPCCPVTCEPSPCQSGCTSSCTPSCCQQSSCQLACCASSPCQQACCVPVCCKTVCCKPVCCVPVCCGDSSCCQQSSCQSACCTSSPCQQACCVPICCKPVCSGISSSCCQQSSCVSCVSSPCCQAVCEPSPCQSGCISSCTPSCCQQSSCQPACCTSSSCQQACCVPVCCKTVCCKPVCSEDSSSCCQQSSCQPACCTSSPCQQACCVPVCCKPVCCKPVGSVPICSGASSLCCQQSSCQPACCTSSQSQQGCCVPVCCKPVSCVPVCSGASSSCCQQSSCQPACCTTSCCRPSSSVSLLCRPVCRPACCVPVPSCCAPTSSCQPSCCRPASCVSLL

SEQ ID NO: 51 - protein, 271 aa, Homo sapiens
MAACTMSVCSSACSDSWRVDDCPESCCEPPCGTAPCLTLVCTPVSCVSSPCCQAACEPSPCQSGCTSSCTPSCCQPACCASSPCQQACCVPVCCKPVCCLPTCSKDSSSCCQQSSCQPTCCASSSCQQSCCVPVCCKPVCCVPTCSEDSSSCCQHSSCQPTCCTSSPCQQSCYVPVCCKPVCFKPICCVPVCSGASTSCCQQSSCQPACCTTSCCRPSSSVSLLCRPICRPACCVPISSCCAPASSYQASCCRPASCVSLLCRPACSRLAC

SEQ ID NO: 52 - protein, 365 aa, Homo sapiens
MAASTMSVCSSDLSYGSRVCLPGSCDSCSDSWQVDDCPESCCEPPCCAPAPCLSLVCTPVSRVSSPCCPVTCEPSPCQSGCTSSCTPSCCQQSSCQLACCASSPCQQACCVPVCCKTVCCKPVCCVSVCCGDSSCCQQSSCQSACCTSSPCQQACCVPVCCKPVCSGISSSCCQQSSCVSCVSSPCCQAVCEPSPCQSGCTSSCTPSCCQQSSCQPTCCTSSPCQQACCVPVCCVPVCCVPTCSEDSSSCCQQSSCQPACCTSSPCQHACCVPVCSGASTSCCQQSSCQPACCTASCCRPSSSVSLLCHPVCKSTCCVPVPSCGASASSCQPSCCRTASCVSLLCRPMCSRPACYSLCSGQKSSC

SEQ ID NO: 53 - protein, 375 aa, Homo sapiens
MAASTMSVCSSDLSYGSRVCLPGSCDSCSDSWQVDDCPESCCEPPCCAPSCCAPAPCLSLVCTPVSRVSSPCCPVTCEPSPCQSGCTSSCTPSCCQQSSCQLACCASSPCQQACCMPVCCKTVCCKPVYCVPVCSGDSSCCQQSSCQSACCTSSPCQQACCVPICCKPVCSGISSSCCQQSSCVSCVSSPCCQAVCEPSPCQSGCISSCTPSCCQQSSCQPACCTSSPCQQACCVPVCCKPVCCVPTCSDDSGSCCQPACCTSSQSQQGCCVPVCCKPVCCVPVCSGASSSCCQQSSCQPACCTTSCCRPSSSVSLLCRPVCRPTCCVPVPSCCAPTSSCQASCCRPASCVSLLCRPACSRPACCGPTSTQKSSC

SEQ ID NO: 54 - protein, 259 aa, Homo sapiens
MADACCTRTYVIAASTMSVCSSDVGHVSRVSSPSTCTGSSWQVDNCQESCCEPRSCASSCCTPSCCAPAPCLALVCAPVSCEPSPCQSGCTDSCTPSCCQQSSCQPACCTSSPCQQACCVPVCCKSNCCKPVCCVSICSGASSPCCQQSSCQSACCTFSPCQQACCVPICCKPICCVPVCSGASSLCCQKSSCQPACCTTSCCRPSSSVSLLCRPVCRPACCVPVPSCCVPASSCQPSCCHPASCLSFLCRPACSRLAC

SEQ ID NO: 55 - protein, 292 aa, Homo sapiens
MAASTMSIRSSAYSDSWQVDDCPESCCEPPCCATSCCAPAPCLTLVCTPVSRVSSPCCQVTCEPSPCQSGCTSSCTPSCCQQSSCQPAYCTSSPCQQACCVPVCCKPVCCVPVCCGASSCCQQSSYQPACCASSSCQPACCVPVCCKPVCCAPTCSEDSYSCCQQSSCQPACCTSSPCQQSYCVPVCCKPVCCKPICCVPVCSGASSLCCQQSGCQPACCTTSCCRPSSSVSLLCRPVCRPACCVPVSSCCAPTSSRQPSYCRQASCVSLLCRPVCSRPACYSFSSGQKSSC

SEQ ID NO: 56 - protein, 251 aa, Homo sapiens
MAASTMSICSSACTDSWRVVDCPESCCEPCCCAPAPSLTLVCTPVSCVSSPCCQTACEPSACQSGYTSSCTTPCYQQSSCQPDCCTSSPCQQACCVPVCCVPVCCVPVCNKPVCFVPTCSESSPSCCQQSSCQPTCCTSSPCQQACCVPVCSKSVCYVPVCSGASTSCCQQSSCQPACCTASCCRPSSSVSLLCHPVCKSTCCVPVPSCGASASSCQPSCCRTASCVSLLCRPVCSRPACYSLCSGQKSSC

SEQ ID NO: 57 - protein, 298 aa, Homo sapiens
MAASTMSVCSSAYSDSWQVDDCPESCCEPPCSAPSCCAPAPSLSLVCTPVSCVSSPCCQAACEPSACQSGCTSSCTPSCCQQSSCQPACCTSSPCQQACCVPVCCKTVCCKPVCCVPVCCGAASSCCRQSSCQPACCASSSCQPACCVPVCCKPVCCVSTCSEDSSSCCQQSSCQPACCTSSSYQQACCVPVCCKTVYCKPICCVPVCSRASSSRCQQPSCQPACCTTSCCRPSSSVSLLCHPVCRSTCCVPVSSCCAPTSSCQSSCCCPASCVSLLCRPASSRLACYSLCSGKKSSC

SEQ ID NO: 58 - protein, 245 aa, Homo sapiens
MSVCSSDLSYGSRVCLPGSCDSCSDSWQVDDCPESCCEPPCCAPAPCLSLVCTPVSRVSSPCCRVTCEPSPCQSGCTSSCTPSCCQQSSCQPACCTSSPCQQACCVPVCCKTVCCKPVCCMPVCCGPSSSCCQQSSCQPACCISSPCQQSCCVPVCCKPICCVPVCSGASSLCCQQSSCQPACCTTSCCRPSSSVSLLCRPVCRPARRVPVPSCCVPTSSCQPSCGRLASCGSLLCRPTCSRLAC

SEQ ID NO: 59 - protein, 163 aa, Homo sapiens
MSFNCSTRNCSSRPIGGRCIVPVAQVTTTSTTDADCLGGICLPSSFQTGSWLLDHCQETCCEPTACQPTCYRRTSCVSNPCQVTCSRQTTCISNPCSTTYSRPLTFVSSGCQPLGGISSVCQPVGGISTVCQPVGGVSTVCQPACGVSRTYQQSCVSSCRRTC

SEQ ID NO: 60 - protein, 96 aa, Homo sapiens
MCHTSCSSGCQPACCAPSPCQASCYIPVGCQSSVCVPVSFKPAVCVPVRCQSSVCVPVSCRPVVYAAPSCQSSGCCQPSCTSVLCRPISCSTPSCC

SEQ ID NO: 61 - protein, 146 aa, Homo sapiens
MCHTSCSSGCQPACCAPSPCQPACCVPSSCQASCCVPVGCQSSVCVPVSFKPAVCLPVSCQSSVCVPMSFKSAVCVPVSCQSSVCVPVSCRPIVCAAPSCQSSLCVPVSCRPVVYAAPSCQSSGCCQPSCTSVLCRPISYSISSCC

SEQ ID NO: 62 - protein, 96 aa, Homo sapiens
MCHTSCSPACQPTCCIRSPCQASCYVPVSCQSSVCMPVSCTRIVCVAPSCQPSVCVPVSCRPIIYVTPSCQSSGCCQPPCTTALCRPISCSTPSCC

SEQ ID NO: 63 - protein, 112 aa, Homo sapiens
MCHTSHSSGCPMACPGSPCCVPSTCYPPEGYGTSCCCSAPCVALLCRPLCGVSTCCQPACCVPSPCQVACCVPVSCKPVLCVASFCPTSGCCQPFCPTLVYRPVTWSTPTGC

SEQ ID NO: 64 - protein, 172 aa, Homo sapiens
MSYNCCSGNFSSRSCGGYLHYPASSCGFSYPSNQVYSTDLCSPSTCQLGSSLYRGCQQTCWEPTSCQTSYVESSPCQTSCYRPRTSLLCSPCQTTYSGSLGFGSSSCRSLGYGSRSCYSVGCGSSGFRSLGYGGCGFPSLGYGVGFCRPTYLASRSCQSSCYRPTCGSGFYY

SEQ ID NO: 65 - protein, 105 aa, Homo sapiens
MGCCPGDCFTCCTQEQNCCEECCCQPGCCGCCGSCCGCGGSGCGGSGCGGSCCGSSCCGSGCGGCGGCGGCGGGCCGSSCCGSSCCGSGCCGPVCCQPTPICDTK

SEQ ID NO: 66 - protein, 88 aa, Homo sapiens
HYGSYYGGLGYSCGGFGGLGYGYGCGCGSFCRRGSGCGYGGYGYGSGFGSYGYGSGFGGYGYGSGFGGYGYGCCRPSYNGGYGFSGFY

SEQ ID NO: 67 - protein, 11 aa, Homo sapiens
  MISC_FEATURE (2)..(2): Xaa = Pro or is not present
  MISC_FEATURE (5)..(5): xaa = Gln, Val, Arg, or Ile
  MISC_FEATURE (10)..(10): Xaa = Gln, Val, Arg, or Ile
SXCCXPSCCXP

SEQ ID NO: 68 - protein, 10 aa, Homo sapiens
  MISC_FEATURE (4)..(4): Xaa = Tyr, Leu, or Phe
YGGXGYGSGY

(19) United States
(12) Patent Application Publication
(10) Pub. No.: US 2006/0286655 A1
(43) Pub. Date: Dec. 21, 2006

(54) Modified KAP polypeptides and use thereof

(75) Inventor: Michel Philippe, Wissous (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/377,338

(22) Filed: Mar. 17, 2006

Related U.S. Application Data
(60) Provisional application No. 60/666,311, filed on Mar. 30, 2005.

(30) Foreign Application Priority Data
Mar. 18, 2005 (FR) 05 50713

(51) Int. Cl. C12N 9/00

(52) U.S. Cl. 435/183

(57) ABSTRACT

Modified polypeptides of the KAP (keratin-associated protein) family and their use in compositions for caring for, treating, strengthening and/or repairing keratin substrates, in particular keratin fibres.

### REFERENCE TO PRIOR APPLICATIONS

This application claims priority to U.S. provisional application 60/666,311 filed Mar. 30, 2005, and to French patent application 0550713 filed Mar. 18, 2005, both incorporated herein by reference.

### FIELD OF THE INVENTION

The field of the invention concerns the use of polypeptides in cosmetic compositions for caring for, strengthening and repairing keratin substrates.

The invention relates especially to modified polypeptides of the KAP (keratin-associated protein) family and to the use thereof in compositions for caring for, treating, strengthening and/or repairing keratin substrates, in particular keratin fibres.

In particular, the invention relates to salified polypeptides or polypeptides modified by covalent bonding of the KAP family, which are suitable for better formulation in cosmetic compositions.

The invention also relates to processes for preparing the modified polypeptides of the KAP family, to cosmetic compositions containing the modified polypeptides, and also to cosmetic processes for caring for, strengthening and/or repairing keratin substrates, using the compositions.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Keratins are fundamental compounds of the skin, the hair, the eyelashes and the nails. These water-insoluble fibrous proteins contribute especially towards their form, elasticity and strength.

In the hair, for example, there are two major types of protein:

the α keratins, which are divided especially into two groups—the acidic keratins (hHa1-8) and the basic to neutral keratins (hHb1-6)—are long-chain helical polypeptides supercoiled in α helices, which form the intermediate microfibrils or filaments of the cortex (FI), inserted in a matrix of KAPs;

the KAPs (Keratin-Associated Proteins) or IFAPs (Intermediate Filament Associated Proteins) form the amorphous matrix between these microfibrils. There are 3 types of KAP divided among 23 families to date: the highly cysteine-rich KAPs (UHS or Ultra High Sulfur, which contain more than 30 mol % of cysteine), the cysteine-rich KAPs (HS or High Sulfur, which contain less than 30 mol % of cysteine) and the glycine- and tyrosine-rich KAPs (HGT or High Glycine and Tyrosine).

These proteins together provide, by means of covalent bonds (especially disulfide bridges), saline bonds, hydrogen bonds or hydrophobic bonds between the amino acid chains, the cohesion of the three-dimensional structure of the hair. In particular, the sequences of the α keratins and of the KAPs have repeating units, one rich in lysine residues, the other in glutamine residues, which constitute potential substrates for enzymes (e.g.: transglutaminases) capable of forming interfilament bridges (FI-FI), interKAP bridges (KAP-KAP) or covalent bonds between the keratin filaments and the KAPs (FI-KAP), playing an essential role in the structure and shape of the hair shaft.

Keratin substrates and in particular keratin fibres may be sensitized or weakened by the chemical action of atmospheric agents (UV, pollution, etc.) and/or treatments (bleaching, dyeing, relaxing, permanent-waving, etc.).

It is known, for example, that sensitized or embrittled hair is often dry, coarse and difficult to disentangle and to style.

It is also known that the nails frequently present structural and consistency defects, the effect of which is to make the surface of the nails unattractive, which may be a source of embarrassment and inconvenience.

L'Oréal patent application WO 99/49837 discloses the use of polyamino acid derivatives for strengthening and caring for keratin fibres.

Patent application WO 03/042387 describes nucleotide and polypeptide sequences of KAP type with hair keratin binding activity, and also cosmetic or therapeutic agents incorporating the sequences.

### SUMMARY OF THE INVENTION

However, to the Applicant's knowledge, no description has ever been given of KAP polypeptides salified or modified by covalent bonding, which are suitable for better formulation in cosmetic compositions for caring for, treating, strengthening and/or repairing keratin substrates, in particular keratin fibres.

### DETAILED DESCRIPTION OF THE PREFERRED EMBODIMENTS

The invention thus relates in several preferred embodiments to polypeptides that are salified or modified by covalent bonding, of the KAP family, functionalized for better formulation in the cosmetic composition, i.e. having good solubility in solvents that are compatible with topical application to keratin substrates and/or good affinity and remanence on the keratin substrates.

The term “keratin substrate” according to the invention covers the skin, the nails and keratin fibres. The term “keratin fibres” means head hair, the eyelashes, the eyebrows and other bodily hair.

The modified polypeptides of the KAP family according to the invention (“modified KAP polypeptides”) are derived from native polypeptides of the KAP family.

Use will be made in particular of polypeptides having a sequence chosen:

(i) the sequences SEQ ID No. 1 to SEQ ID No. 66;

(ii) a fragment of the sequences comprising at least 3 consecutive amino acids of a repeat sequence chosen from the sequence SZCCXPSCCXP (Z: Ø, P and X: Q, V, R, I) and the sequence YGGXGYGSGY (X: Y, L, F); and

(iii) homologues thereof.

“Z” may correspond to a proline (P) amino acid or to no amino acid (Ø).

The sequences SEQ ID No. 1 to SEQ ID No. 66 are given in the appendix, attached hereto and incorporated herein by reference.

The fragments (ii) comprising at least 3 consecutive amino acids of a repeat sequence chosen from the sequence SZCCXPSCCXP (Z: Ø, P and X: Q,V,R,I) and the sequence YGGXGYGSGY (X:Y,L,F) will advantageously be potential substrates of enzymes (e.g.: transglutaminases) capable of forming interfilament bridges (FI-FI), interKAP bridges (KAP-KAP) or covalent bonds between the keratin filaments and KAPs (FI-KAP).

In particular, the fragment containing at least 3 consecutive amino acids of the repeat sequence SZCCXPSCCXP (Z: Ø, P and X: Q,V,R,I) and the sequence YGGXGYGSGY (X:Y,L,F), preferably of at least 4 amino acids and even more preferentially of at least 5 consecutive amino acids of the repeat sequence, will comprise at least one amino acid capable of forming covalent bonds (e.g.: cysteine), saline bonds (e.g.: lysine, arginine, histidine, aspartate or glutamate), hydrogen bonds (e.g.: serine or tyrosine) or hydrophobic bonds (e.g.: glycine, alanine, valine, leucine or isoleucine), either with the keratin intermediate filaments (FI) or with other KAPs, or alternatively with other constituent proteins of the hair.

The fragment (ii) may contain from 3 to 60 amino acids, in particular from 3 to 20 amino acids and even more preferentially from 3 to 10 amino acids and will also be wherein it has at least one amino acid that is salifiable and/or modifiable by covalent bonding, chosen from the amino acids lysine, histidine, arginine, aspartate, glutamate, cysteine, methionine, tyrosine, threonine and serine.

This polypeptide fragment may be obtained by proteolysis or synthetically according to the known methods.

According to one particular embodiment, the following may be used:

the sequence SEQ ID No. 9 (access No. Q9BYS0), which contains salifiable amino acids (arginine) and amino acids that are modifiable by covalent bonding (threonine, cysteine, serine and arginine), or

a fragment of this sequence SEQ ID No. 9, preferably the fragment SPCCR.

According to the invention, the term “homologues” means any peptide sequence that is at least 50%, preferably at least 80% and even more preferentially at least 95% identical to the peptide sequence (i) chosen from the sequences SEQ ID No. 1 to SEQ ID No. 66 or to the fragment (ii) as defined above, in the same species or in a different species: in the latter case, it is also referred to as an “orthologous polypeptide”.

The term “percentage of identity” between two peptide sequences or amino acid sequences is intended to denote a percentage of amino acid residues that are identical between the two sequences to be compared, obtained after the best alignment, i.e. the optimum alignment achieved, for example, using the Smith-Waterman local homology algorithm (1981, Ad. App. Math. 2: 482), using the Neddleman-Wunsch local homology algorithm (1970, J. Mol. Biol. 48: 443), using the Pearson-Lipman similarity search method (1988, Proc. Natl. Acad. Sci. USA 85:2444), or using computer software using these algorithms (GAP, BESTFIT, BLAST P and BLAST N available on the site NCBI, FASTA and TFASTA in Wisconsin Genetics Software Package, Genetics Computer Group, 575 Science Dr, Madison, Wis.).

The original KAP polypeptides may be polypeptides of natural or synthetic origin.

The term “natural origin” means a polypeptide in pure form or dissolved at various concentrations, obtained via various extraction processes from a keratin material (skin, nail or hair, in particular hair) of natural origin.

The term “synthetic origin” means a polypeptide in pure form or dissolved at various concentrations, obtained chemically or via production in an organism after introducing into this organism the components required for this production. For example, the polypeptide will be a recombinant polypeptide.

These polypeptides of the KAP family will be modified according to the invention via a chemical and/or enzymatic process comprising at least one reaction chosen from a salification reaction or a derivatization reaction (e.g.: nucleophilic substitution or redox reaction or acylation) as described below.

The invention thus relates to a modified polypeptide of the KAP family (“modified KAP polypeptide”), wherein it contains at least one salified amino acid and/or one amino acid modified by covalent bonding. In preferred embodiments, the invention modified KAP polypeptides are isolated and/or purified and thus in a different form from any such polypeptide potentially existing in nature.

In particular, the modified polypeptide of the KAP family has a peptide sequence chosen from (i) the sequences SEQ ID No. 1 to SEQ ID No. 66, (ii) a fragment of the sequences comprising at least 3 consecutive amino acids of a repeat sequence chosen from the sequence SZCCXPSCCXP (Z: Ø, P and X: Q, V, R, I) and the sequence YGGXGYGSGY (X: Y, L, F) and (iii) homologues thereof, and contains at least one salified amino acid and/or one amino acid modified by covalent bonding.

In particular, the modified KAP polypeptide may be obtained from the native sequence SEQ ID No. 9 (access No. Swissprot Q9BYS0) or from a fragment of this sequence according to the invention, in particular the fragment SPCCR.

According to the invention, the term “amino acid modified by covalent bonding” means an amino acid modified, for example, by a substitution reaction, a redox reaction or an acylation reaction.

In particular, the modified polypeptide of the KAP family according to the invention contains at least one amino acid that is salified and/or modified by covalent bonding and up to 100% of amino acids that are salified and/or modified by covalent bonding relative to the total number of amino acids that are salifiable and/or modifiable by covalent bonding of the native polypeptide, preferably from 20% to 80% and ever more preferentially from 30% to 60% of amino acids that are salified and/or modified by covalent bonding relative to the total number of amino acids that are salifiable and/or modifiable by covalent bonding of the native polypeptide.

“Salifiable amino acids” according to the invention that may be mentioned include the amino acids lysine, histidine, arginine, aspartate and glutamate.

As “amino acids that are modifiable by covalent bonding” according to the invention, mention may be made of the amino acids cysteine, methionine, lysine, histidine, arginine, aspartate, glutamate, tyrosine, threonine and serine.

According to a first aspect of the invention, the modified polypeptide of the KAP family according to the invention is a cationic polypeptide.

It may be obtained by adding at least one acid chosen from hydrochloric acid, acetic acid, succinic acid, palmitic acid, stearic acid, oleic acid, behenic acid, 18-methyleicosanoic acid, α-hydroxy acids and β-hydroxy acids, and mixtures thereof.

According to another aspect of the invention, the modified polypeptide of the KAP family according to the invention is an anionic polypeptide.

It may be obtained by adding at least one base chosen from mineral bases, guanidine derivatives, primary amines and tertiary amines, and mixtures thereof.

According to another aspect of the invention, the modified polypeptide of the KAP family according to the invention is a polypeptide chemically and/or enzymatically modified by covalent bonding. It may be obtained especially by adding electrophilic or nucleophilic derivatives such as a carboxylic acid derivative, a thiol derivative or an amine derivative (“carboxylic acid compound, a thiol compound or an amine compound”).

The invention also relates to processes for preparing the modified polypeptides of the KAP family according to the invention.

According to a first aspect, it is a process for preparing a salified polypeptide of the KAP family comprising at least one step of salification of a KAP polypeptide in the presence of an acid or a base.

The acid or the base reacts specifically with the side chains of the KAP polypeptide comprising ionizable amine or acid groups (e.g.: lysine, histidine, arginine, aspartate or glutamate).

This reaction is generally performed at a temperature ranging from 0° C. to 60° C. and preferably between 20° C. and 45° C., regulated at a given pH ranging from 5 to 9 according to the structure and the solubility of the polypeptide of the KAP family.

The reaction time may range from 15 minutes to 3 hours depending the general structural of the polypeptide and on the number of salifiable amino acids present in the polypeptide.

According to one particular embodiment, the reaction is assisted by dissolution in ultrasound baths according to the standard technique.

Preferably, the reaction will be performed in an aqueous base, in the presence of hydrophilic organic solvents, such as linear or branched lower monoalcohols containing from 1 to 8 carbon atoms, for instance ethanol, propanol, butanol, isopropanol, isobutanol, optionally oxyethylenated polyethylene glycols, polyols such as propylene glycol, isoprene glycol, butylene glycol, glycerol, sorbitol and derivatives thereof, and propylene glycol ethers.

However, it may also be performed in or in the presence of an oily base comprising oils such as hydrocarbon-based oils of animal origin such as perhydrosqualene; hydrocarbon-based plant oils such as liquid triglycerides of fatty acids containing from 4 to 10 carbon atoms, for instance heptanoic or octanoic acid triglycerides, or alternatively sunflower oil, corn oil, soybean oil, grapeseed oil, sesame seed oil, apricot oil, macadamia oil, castor oil, avocado oil, caprylic/capric acid triglycerides, jojoba oil or shea butter; linear or branched hydrocarbons, of mineral or synthetic origin, such as liquid paraffins and derivatives thereof, petroleum jelly, polydecenes, and hydrogenated polyisobutene such as parleam; synthetic esters and ethers, especially of fatty acids, for instance purcellin oil, isopropyl myristate, 2-ethyl-hexyl palmitate, 2-octyldodecyl stearate, 2-octyl-dodecyl erucate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates or decanoates; polyol esters, for instance propylene glycol dioctanoate, neopentyl glycol diheptanoate or diethylene glycol diisononanoate; and pentaerythritol esters; fatty alcohols containing from 12 to 26 carbon atoms, for instance octyldodecanol, 2-butyloctanol, 2-hexyl-decanol, 2-undecylpentadecanol or oleyl alcohol; partially hydrocarbon-based and/or partially silicone-based fluoro oils; silicone oils, for instance volatile or non-volatile, linear or cyclic polymethylsiloxanes (PDMS) that are liquid or pasty at room temperature, for instance cyclomethicones, dimethicones, optionally comprising a phenyl group, for instance phenyl trimethicones, phenyltrimethylsiloxydiphenylsiloxanes, diphenylmethyldimethyltrisiloxanes, diphenyl dimethicones, phenyl dimethicones and polymethylphenylsiloxanes; mixtures thereof.

The reaction may also be performed in an emulsified base using an aqueous phase and an oily phase.

The salified polypeptides obtained may be used in unmodified form, without an additional purification step.

In particular, a preferred process will comprise the following steps:

(a) a polypeptide of the KAP family having a peptide sequence chosen from SEQ ID No. 1 to SEQ ID No. 66, or a fragment of the sequences comprising at least 3 consecutive amino acids of a repeat sequence chosen from the sequence SZCCXPSCCXP (Z: Ø, P and X: Q, V, R, I) and the sequence YGGXGYCSGY (X: Y, L, F) and (b) at least one compound chosen from an acid and a base are reacted, in an aqueous, oily or emulsified base;

the temperature is adjusted to between 20° C. and 40° C. and the pH to between 5 to 9; and

the reaction is continued for a time ranging from, e.g., 15 minutes to 3 hours.

In particular, the sequence SEQ ID No. 9 or a fragment of this sequence, preferably the fragment SPCCR, will be used.

An acid preferably chosen from hydrochloric acid, acetic acid, succinic acid, palmitic acid, stearic acid, oleic acid, behenic acid, 18-methyleicosanoic acid, α-hydroxy acids (e.g.: glycolic acid, lactic acid or citric acid) and β-hydroxy acids (e.g.: salicylic acid), and mixtures thereof, will be used to obtain a cationic polypeptide.

Hydrochloric acid, acetic acid, succinic acid, α-hydroxy acids or β-hydroxy acids, for instance citric acid, or mixtures thereof, will preferentially be used to obtain a cationic polypeptide suitable for an aqueous formulation.

Palmitic acid, stearic acid, oleic acid, behenic acid or 18-methyleicosanoic acid, or mixtures thereof, will preferentially be used to obtain a cationic polypeptide suitable for an oily formulation.

A base preferably chosen from mineral bases (e.g.: sodium hydroxide, potassium hydroxide or bicarbonate), organic bases (e.g.: ammonia), guanidine derivatives (e.g.: guanidinium hydroxide, tetramethylguanidine), primary amines (e.g.: glucamine) and tertiary amines (e.g.: triethanolamine, N,N-diethylethanolamine, N-methyldiethanolamine), and mixtures thereof, will be used to obtain an anionic polypeptide.

Another aspect of the invention relates to a chemical and/or enzymatic process for preparing a polypeptide modified by covalent bonding, of the KAP family, comprising at least one step of derivatization of a polypeptide of the KAP family.

The derivatization reaction consists, for example, of a modification, with formation of a covalent bond, of any reactive functional group located on the side chains (e.g., NH2, COOH, OH, SH, S—CH3, arginyl residue or histidyl residue).

In particular, it will be sought to modify the thiol-containing amino acids of the KAP polypeptide (e.g.: cysteine or methionine), the cationic amino acids (e.g.: lysine, histidine or arginine), the anionic amino acids (e.g.: aspartate or glutamate) and/or the amino acids containing a hydroxyl function (e.g.: tyrosine, threonine or serine).

Even more preferentially, modifications on cysteine, lysine, histidine, arginine, serine and tyrosine will be preferred.

These modifications via derivatization reactions are well known to those skilled in the art: they are described especially in “Techniques in protein modification” by R. L. Lundblad, pp. 63 to 268, 1994, CRC Press, in “Chemistry of the Amino acids” by J. P. Greenstein and M. Winitz, vol. 2, pp. 883 to 1268, Krieger Publishing Company, reprint edition 1984, or in “The Chemical Synthesis of Peptides” by J. Jones, pp. 17 to 94, Oxford Science Publications, 1991.

Examples of derivatization reactions that may especially be mentioned include:
a) substitution or acylation reactions via the nucleophilic functions of KAPs (e.g.: cysteine, lysine, tyrosine, histidine or serine) on any electrophile and in particular on the carboxylic acid derivatives (e.g.: esters, anhydride, halides including chlorides, and imidazolides) and sulfonic acid derivatives (e.g.: chlorides);
b) oxidation reaction (if free SH) or reduction reaction (if SS) of cysteines of KAP polypeptides, for example by reaction with a thiol derivative or with any other reducing or oxidizing agent.

These reactions are generally performed at a temperature ranging from room temperature to the boiling point of the reaction solvent.

In particular, a reaction at a temperature ranging from 20 to 180° C. and preferably from 20 to 80° C. may be used. Standard heating or heating with microwave energy may be used.

The reaction time is generally from 30 minutes to 8 hours depending on the structure of the polypeptide (size, solubility and number of substitutable amino acids).

The reaction may be performed in an aqueous base, an oily base, an emulsified base as described above for the salification reaction, or in a solvent-free medium.

For example, the process for preparing a polypeptide modified by covalent bonding, of the KAP family, via a substitution or acylation reaction can comprise the following steps:

(a) a polypeptide of the KAP family having a peptide sequence chosen from SEQ ID No. 1 to SEQ ID No. 66, or a fragment of the sequences comprising at least 3 consecutive amino acids of a repeat sequence chosen from the sequence SZCCXPSCCXP (Z: Ø, P and X: Q, V, R, I) and the sequence YGGXGYCSGY (X: Y, L, F) and (b) at least one carboxylic acid derivative are reacted, in an aqueous, oily or emulsified base or in a solvent-free medium;

the temperature is adjusted to between 20° C. and 180° C.;

the reaction is continued for a time ranging from 30 minutes to 8 hours.

By way of example, the sequence SEQ ID No. 9 or a fragment of this sequence, preferably the fragment SPCCR, may be used.

The substitution or acylation reaction may advantageously be catalyzed in the presence of a base. Preferably, a tertiary amine (e.g.: triethanolamine, N,N-diethylethanolamine or N-methyldiethanolamine) will be used.

An anhydride derivative (e.g.: succinic anhydride) will be used, for example, to obtain a KAP polypeptide modified by covalent bonding that is suitable for dissolution in aqueous solvents.

A fatty acid chloride (e.g.: olely chloride) can be used, for example, to obtain a KAP polypeptide modified by covalent bonding that is suitable for dissolution in oils.

According to another embodiment, the process for preparing a polypeptide modified by covalent bonding, of the KAP family, via a redox reaction can comprise the following steps:

(a) a polypeptide of the KAP family having a peptide sequence chosen from SEQ ID No. 1 to SEQ ID No. 66, or a fragment of the sequences comprising at least 3 consecutive amino acids of a repeat sequence chosen from the sequence SZCCXPSCCXP (Z: Ø, P and X: Q, V, R, I) and the sequence YGGXGYCSGY (X: Y, L, F) and (b) at least one thiol derivative are reacted, in an aqueous, oily or emulsified base or in a solvent-free medium;

the temperature is adjusted to between 20° C. and 80° C.;

the reaction is continued for a time ranging from 30 minutes to 8 hours.

By way of example, the sequence SEQ ID No. 9, or a fragment of this sequence, preferably the fragment SPCCR, may be used.

Thioglycolic acid can be used, for example, to obtain a KAP polypeptide modified by covalent bonding that is suitable for dissolution in aqueous solvents.

An octadecane thiol can be used, for example, to obtain a KAP polypeptide modified by covalent bonding that is suitable for dissolution in oils.

The modified polypeptides of the KAP family according to the invention show good solubility in aqueous and/or alcoholic and/or oily solvents, i.e. a solubility at least equal to that of the original polypeptides of the KAP family (unmodified), and preferably a higher solubility, for example 10% to 15% higher than the solubility of the unmodified polypeptides of the KAP family.

According to the invention, the term “solubility” in solvents means the capacity of a polypeptide, mixed with one or more solvents, to dissolve in the solvent(s).

This solubility may be evaluated according to the techniques known to those skilled in the art.

For example, the principle may entail mixing the polypeptide and an aqueous and/or alcoholic and/or oily solvent, and in performing a macroscopic observation after a given time. Thus, the presence or absence of crystals and/or of a deposit in the solution is measured, for example after one week, which makes it possible to define for each polypeptide the aqueous and/or alcoholic and/or oily solvent that is the most suitable for total dissolution of the polypeptide.

The modified polypeptides of the KAP family according to the invention also show good affinity for and remanence on the keratin substrate, in particular on the keratin fibre, or even a higher affinity and remanence, for example from 10% to 15% higher than that of the original (unmodified) polypeptides of the KAP family.

According to the invention, the term “affinity” for the keratin substrate means the capacity of a polypeptide to bind to the keratin substrate at the time of application of the composition to the keratin substrate.

According to the invention, the term “remanence” on the keratin substrate means the capacity of a polypeptide to remain bound to the keratin substrate after application of the composition to the keratin substrate, in particular after washing or shampooing one or more times.

The affinity and remanence of the polypeptides for and on the keratin fibre may be conventionally measured by UV assay (e.g.: mass spectrophotometry), which makes it possible to define the amount of polypeptide bound or fixed to the keratin fibre.

The principle may entail, for example, depositing the polypeptide or a composition containing the peptide onto the lock, leaving it to act for a given time, and then rinsing the lock, washing it with a shampoo, rinsing it again and drying it with a hairdryer.

Different portions of hair are cut in the middle of the hair and introduced into a mass spectrophotometer. The results are expressed as a ratio of the mass of polypeptide bound to the hair to the mass of hair. The percentage of polypeptide bound to the lock at the time of deposition (affinity), or after a given number of shampoo washes (remanence), may thus be measured.

The affinity and the remanence may also be conventionally measured by optical microscopy, at the time of deposition and after a given number of shampoo washes, for example after 5 shampoo washes. The deposit on the lock is thus evaluated qualitatively.

The invention also relates to the cosmetic use of at least one modified polypeptide of the KAP family as defined according to the invention, in a cosmetic composition comprising a physiologically acceptable medium.

In particular, a KAP polypeptide that is salified and/or modified by covalent bonding obtained according to one of the techniques described above using SEQ ID No. 9 or a fragment of this sequence, preferably the fragment SPCCR, may be used.

According to the invention, the term “physiologically acceptable medium” means a cosmetically acceptable medium that is compatible with keratin substrates.

In general, a medium that is compatible with keratin substrates may be anhydrous or aqueous: it may thus comprise an aqueous phase and/or a fatty phase, comprising at least one fatty substance chosen from oils, waxes and pasty fatty substances, and mixtures thereof.

The term “aqueous phase” means water or a mixture of water and of hydrophilic organic solvent(s), for instance alcohols and especially linear or branched lower monoalcohols containing from 2 to 5 carbon atoms, for instance ethanol, isopropanol or n-propanol, and polyols, for instance glycerol, diglycerol, propylene glycol, sorbitol, pentylene glycol and polyethylene glycols, or alternatively hydrophilic C₂ ethers and C₂-C₄ aldehydes. Water or a mixture of water and of hydrophilic organic solvents may be present in the composition according to the invention in a content ranging from 0.1% to 99% by weight and preferably from 10% to 80% by weight relative to the total weight of the composition.

The term “fatty phase” means a phase comprising at least one fatty substance, chosen from oils, waxes and pasty fatty substances, and mixtures thereof.

In particular, the modified polypeptide of the KAP family is intended for:

maintaining and/or restoring and/or improving the protein content of the keratin substrate;

maintaining and/or restoring and/or improving the surface state of the keratin substrates;

conserving and/or restoring and/or improving the physical and/or mechanical properties of the keratin substrates.

It will thus be intended especially for improving the softness of keratin fibres, and/or their suppleness and/or elasticity and/or resistance to breaking and/or volume and/or hairstyle hold and/or resistance to chemical treatments.

The modified polypeptide of the KAP family according to the invention is present in the composition in any amount, and is preferably present in a sufficient amount depending on the desired effect. In particular, this amount may range from 0.001% to 30% by weight relative to the total weight of the composition, preferably from 0.01% to 15% and even more preferentially from 0.1% to 5% by weight relative to the total weight of the composition.

The invention also relates to a composition comprising, in a physiologically acceptable medium suitable for topical application to keratin substrates, at least one modified polypeptide of the KAP family as defined above.

When the medium of the composition predominantly comprises aqueous solvents, at least one modified polypeptide of the KAP family that may be obtained via a chemical process including at least one step of salification by addition of at least one acid chosen from hydrochloric acid, acetic acid, citric acid and succinic acid, and mixtures thereof, will preferentially be used.

When the medium of the composition predominantly comprises oily solvents, at least one modified polypeptide of the KAP family that may be obtained via a chemical process including at least one step of salification by addition of at least one acid chosen from palmitic acid, stearic acid, oleic acid, behenic acid and 18-methyleicosanoic acid, and mixtures thereof, will preferentially be used.

In particular, the modified polypeptide of the KAP family is present in the composition in any amount, including in an amount ranging from 0.001% to 30% by weight relative to the total weight of the composition, preferably from 0.01% to 15% and even more preferentially from 0.1% to 5% by weight relative to the total weight of the composition.

The composition according to the invention may also comprise at least one cosmetic ingredient, covalently or non-covalently associated with the modified polypeptide of the KAP family.

This cosmetic ingredient may be any ingredient known in the cosmetic field.

In particular, the cosmetic ingredient may be chosen from a conditioning agent, a care agent and/or a treating agent for keratin substrates, a cosmetic adjuvant or a formulating agent, and mixtures thereof.

The conditioning, care and/or treating agents for keratin substrates are especially intended to improve the appearance and/or the surface state of the keratin substrates (e.g.: softer, smoother, less split hair, shinier nails, etc.), to preserve and/or to improve the physical and/or mechanical properties of keratin substrates (e.g.: stronger hair, which is easier to style, less brittle nails, etc.), in particular when the keratin substrates are sensitized or weakened by the chemical action of atmospheric and/or treating agents.

Examples of conditioning agents for keratin substrates that may be mentioned include oils, waxes, ceramides, gums, silicones, polysiloxanes, polymers, film-forming polymers and fixing polymers, and mixtures thereof.

As agents for caring for and/or treating keratin substrates, mention may be made especially of moisturizers, agents for stimulating the synthesis of dermal or epidermal macromolecules and/or preventing their degradation, anti-pollution agents or free-radical scavengers, softeners, vitamins, amino acids, proteins, antidandruff agents, anti-seborrhoeic agents, reducing agents, hair dye precursors, direct hair dyes, oxidizing agents, agents for promoting curling of the eyelashes, agents for promoting nail growth, nail hardeners, etc.

As cosmetic adjuvants or formulating agents conventionally used in cosmetic compositions, mention may be made of organic solvents, surfactants, plasticizers, thickeners, emulsifiers, preserving agents, UV-screening agents, fillers, dyestuffs, fragrances, opacificiers, humectants, agents for adjusting and fixing the pH, antibacterial agents, antifungal agents and propellants, and mixtures thereof.

A person skilled in the art will adapt the amount of cosmetic ingredient in the composition as a function of the desired effect and such that the amount of cosmetic ingredient present in the composition does not affect or affect too much the desired properties of the modified polypeptide of the KAP family.

In particular, the cosmetic ingredient will be present in the composition in any amount including in an amount that may range from 0.001% to 20% by weight relative to the total weight of the composition, preferably from 0.01% to 10% and even more preferentially from 0.1% to 2% by weight relative to the total weight of the composition.

The compositions according to the invention comprising, in a physiologically acceptable medium, at least one modified polypeptide of the KAP family may be in any form including in any known galenical form that is suitable for topical application to keratin substrates (skin, eyelashes, nails or hair).

A person skilled in the art may choose the appropriate form, and also the method for preparing it, on the basis of his general knowledge, taking into account firstly the nature of the constituents used, especially their solubility in the support, and secondly the intended use of the composition.

The compositions suitable for topical application according to the invention may especially be in the form of aqueous, aqueous-alcoholic or oily solutions, oil-in-water (O/W), water-in-oil (W/O) or multiple (W/O/W or O/W/O) emulsions, aqueous or oily gels, dehydrated anhydrous products, or dispersions of an oily phase in an aqueous phase using spherules, these spherules possibly being polymer nanoparticles such as nanospheres and nanocapsules, or lipid vesicles of ionic type (liposomes) and/or nonionic type.

When the composition used according to the invention is an emulsion, the proportion of the fatty phase may preferably range from 5% to 80% by weight and preferably from 5% to 50% by weight relative to the total weight of the composition. The oils, the emulsifiers and the coemulsifiers used in the composition in emulsion form are chosen from those conventionally used in the field under consideration. The emulsifier and the coemulsifier are present in the composition in a proportion preferably ranging from 0.3% to 30% by weight and preferably from 0.5% to 20% by weight relative to the total weight of the composition.

For application in particular to the hair, the composition may be in the form of creams, lotions, gels, emulsions or mousses or in the form of aerosol compositions also comprising a pressurized propellant. It may be in the form of a haircare lotion, for example for daily or twice-weekly application, a shampoo or hair conditioner, in particular for twice-weekly or weekly application, a liquid or solid scalp cleansing soap for daily application, a hairstyle shaping product (lacquer, hairsetting product, styling gel or mousse), a colouring shampoo, a permanent-waving composition, a treating mask, or a foaming cream or gel for cleansing the hair. It may also be in the form of a hair dye or hair mascara to be applied with a brush or a comb.

For application to the eyelashes, the composition will preferably be in the form of a mascara that may be an eyelash makeup composition, an eyelash makeup base, a composition to be applied over a mascara, also known as a top coat, or a cosmetic eyelash treatment composition. The mascara is more particularly intended for human eyelashes, but also false eyelashes.

For application to the nails, the composition may be a nail makeup product such as a coloured nail varnish, a nail base or “base coat”, a finishing composition or “top coat”, a composition to be applied under or over the nail makeup product, a nail varnish dissolver or a cosmetic nailcare product such as a treating base for protecting, strengthening and/or repairing the nails.

For application to the skin, the composition may be more or less fluid and may have the appearance of a white or coloured cream, an ointment, a milk, a lotion, a serum, a paste or a mousse. It may also be in solid form, in particular in the form of a stick. It may be used as a care product and/or as a makeup product for the skin.

The compositions will be applied by any suitable means, such as a fine brush, a coarse brush, a spray or with the fingers, for example.

The invention also relates to a cosmetic process for caring for, treating, strengthening and/or repairing keratin substrates, wherein a composition as defined above is applied to the skin, the hair, the eyelashes or the nails, optionally followed by rinsing.

According to the invention, the term “care of keratin substrates” means a composition intended to improve the appearance and/or the surface state of keratin substrates. The care of keratin substrates may consist in making the hair softer, smoother and less split; and in making the nails less brittle and less split.

According to the invention, the term “strengthening and/or repairing keratin substrates” means a composition intended to conserve and/or restore and/or improve the physical and/or mechanical properties of keratin substrates, which may be manifested, for example:

either by rigidification of the keratin substrates, which gives them greater consistency and body, and also a more pleasant feel. This results, for example, in an increase in the volume of the keratin fibres and also greater ease of styling and styling hold;

or by better elasticity and/or better resistance to mechanical tensile forces applied thereto, for example during combing for keratin fibres, in particular on African hair or any embrittled or sensitized hair.

The hair is thus stronger, more supple, softer, smoother and easier to style. The nails are thus smoother, shinier, less damaged, less split and less brittle.

According to a first embodiment, the composition may be applied as a pre-treatment to a treatment liable to sensitize keratin substrates (e.g.: before oxidation dyeing).

According to another embodiment, the composition may be applied as a post-treatment to a treatment liable to sensitize keratin substrates (e.g.: after a peeling treatment).

The composition containing the modified polypeptide of the KAP family may also constitute the treating composition (e.g.: combined with a dye).

The application of the compositions according to the process of the invention will be performed according to the usual technique for using the compositions. For example:

the application to the hair may be performed before (lotion, 1 hour before), during (shampoo) or after (spray lotions) shampooing; the composition may be applied to dry hair (lacquers, sprays or lotions) or to wet hair (permanent-reshaping or hairsetting compositions);

the application of a care base to the nails may be performed before or after applying varnish;

the application of a care composition for the eyelashes may be performed immediately after applying mascara;

the application of a skincare composition may be performed immediately after applying a cleansing solution.

In particular, the process according to the invention will be intended for caring for, treating, strengthening and/or repairing sensitized keratin substrates, and preferentially sensitized hair.

The term “sensitized hair” means hair that has been embrittled or damaged by hair treatments, for instance relaxing, permanent-waving or dyeing operations, or by the effect of atmospheric agents. Sensitized hair is coarse after washing and drying, has poorer mechanical behaviour, more static electricity and looks dull.

The compositions may be applied daily or at a frequency of 2 to 3 applications per week, for a duration of 1 to 3 months, which may be renewed according to the degree of impairment of the individual's keratin substrates to be treated.

The compositions according to the invention will thus be intended to improve the appearance and/or surface state of keratin substrates, in particular to make the hair softer, smoother, less split and more resistant to breaking, to give it greater consistency and body, especially more volume and more hairstyle hold, more elasticity and suppleness, and also greater resistance to treatments liable to weaken it (e.g.: dyeing or permanent-waving).

The invention is illustrated in greater detail in the non-limiting example that follows:

### EXAMPLE

### Preparation of a Modified KAP Polypeptide of Cationic Type

The fragment SPCCR of the sequence SEQ ID No. 9 (access No. Q9BYS0) corresponding to a fragment of repeat sequence SZCCX (Z: P and X: R) containing an arginine amino acid (salifiable) and two cysteine amino acids, is chosen as native sequence.

This sequence fragment may be obtained via chemical synthesis according to the standard techniques.

A salification reaction is then performed, which entails:

placing the sequence fragment to be reacted in an aqueous base in the presence of an ethanol/propylene glycol and hydrochloric acid mixture;

adjusting the temperature to 40° C. and the pH to a minimum of 5;

continuing the reaction for a time that is sufficient to salify the arginine present in the sequence, for example about 1 hour.

The cationic polypeptide obtained is extracted from the solution by salification; it may be used in unmodified form or may undergo a further purification step.

This polypeptide is particularly suitable for incorporation into a cosmetic formulation of aqueous type.

According to one alternative aimed at obtaining a polypeptide that is particularly suitable for incorporation into a cosmetic formulation of oily type, an emulsified base is used and the hydrochloric acid is replaced with oleic acid in the process described above.

The invention modified polypeptides, compositions and methods are preferably used by subjects desirous of the benefits noted herein, subjects “in need of” these benefits. One using the invention as disclosed will preferably use an amount of the invention modified polypeptides and compositions effective to produce the desired benefit(s). Such amount is inclusive of an amount of the polypeptides and compositions described herein at the disclosed concentrations of active ingredients sufficient to cover the keratin being treated in a single application, and also includes an amount applied upon repeated application, for example on a daily basis over a course of days, weeks, etc. In a preferred embodiment the invention process includes multiple applications of the invention polypeptides and composition to keratin materials in need of attention.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A modified KAP polypeptide comprising at least one salified amino acid and/or one amino acid modified by covalent bonding.

2. The polypeptide according to claim 1, having a sequence chosen from (i) the sequences SEQ ID No. 1 to SEQ ID No. 66, (ii) a fragment of the sequences comprising at least 3 consecutive amino acids of a repeat sequence chosen from the sequence SZCCXPSCCXP (Z: Ø, P and X: Q, V, R, I) and the sequence YGGXGYGSGY (X: Y, L, F) and (iii) homologues thereof.

3. The polypeptide according to claim 2, having from 3 to 60 amino acids.

4. The polypeptide according to claim 1, wherein it comprises from 1% to 100% of amino acids that are salified and/or modified by covalent bonding relative to the total number of amino acids that are salifiable and/or modifiable by covalent bonding of the native polypeptide.

5. The polypeptide according to claim 1, wherein the amino acids that are salified and/or modified by covalent bonding are chosen from amino acids lysine, histidine, arginine, aspartate, glutamate, cysteine, methionine, tyrosine, threonine and serine.

6. The polypeptide according to claim 1, wherein it is a cationic polypeptide.

7. The polypeptide according to claim 1, wherein it is an anionic polypeptide.

8. The polypeptide according to claim 1, having a peptide sequence SEQ ID No. 9 or a fragment SPCCR thereof.

9. A process for preparing a salified KAP polypeptide, comprising:
(a) a KAP polypeptide having a peptide sequence chosen from SEQ ID No. 1 to SEQ ID No. 66, or a fragment of the sequences comprising at least 3 consecutive amino acids of a repeat sequence chosen from the sequence SZCCXPSCCXP (Z: Ø, P and X: Q, V, R, I) and the sequence YGGXGYGSGY (X: Y, L, F) and (b) at least one compound chosen from an acid and a base are reacted, in an aqueous, oily or emulsified base;
the temperature is adjusted to between 20° C. and 40° C. and the pH to between 5 to 9; and
the reaction is continued for a time of from 15 minutes to 3 hours.

10. The process according to claim 9, wherein acid is a reactant and the acid is chosen from hydrochloric acid, acetic acid, succinic acid, palmitic acid, stearic acid, oleic acid, behenic acid, 18-methyleicosanoic acid, α-hydroxy acids and β-hydroxy acids, and mixtures thereof.

11. The process according to claim 9, wherein base is a reactant and the base is chosen from mineral bases, organic bases, guanidine derivatives, primary amines and tertiary amines, and mixtures thereof.

12. A process for preparing a KAP polypeptide modified by covalent bonding via a substitution or acylation reaction comprising:
(a) a KAP polypeptide having a peptide sequence chosen from SEQ ID No. 1 to SEQ ID No. 66, or a fragment of the sequences comprising at least 3 consecutive amino acids of a repeat sequence chosen from the sequence SZCCXPSCCXP (Z: Ø, P and X: Q, V, R, I) and the sequence YGGXGYGSGY (X: Y, L, F) and (b) at least one carboxylic acid compound are reacted, in an aqueous, oily or emulsified base or in a solvent-free medium;
the temperature is adjusted to between 20° C. and 180° C.;
the reaction is continued for a time of from 30 minutes to 8 hours.

13. A process for preparing a KAP polypeptide modified by covalent bonding comprising:
(a) a KAP polypeptide having a peptide sequence chosen from SEQ ID No. 1 to SEQ ID No. 66, or a fragment of the sequences comprising at least 3 consecutive amino acids of a repeat sequence chosen from the sequence SZCCXPSCCXP (Z: Ø, P and X: Q, V, R, I) and the sequence YGGXGYGSGY (X: Y, L, F) and (b) at least one thiol compound are reacted, in an aqueous, oily or emulsified base or in a solvent-free medium;
the temperature is adjusted to between 20° C. and 80° C.;
the reaction is continued for a time of from 30 minutes to 8 hours.

14. The process according to claim 9, wherein the peptide sequence SEQ ID No. 9 or a fragment SPCCR thereof is a reactant.

15. The process according to claim 12, wherein the peptide sequence SEQ ID No. 9 or a fragment SPCCR thereof is a reactant.

16. The process according to claim 13, wherein the peptide sequence SEQ ID No. 9 or a fragment SPCCR thereof is a reactant.

17. A composition comprising, in a physiologically acceptable medium suitable for topical application to keratin substrates, at least one modified KAP polypeptide as defined in claim 1.

18. The composition according to claim 17, wherein the modified KAP polypeptide is present in the composition in an amount of 0.001% to 30% by weight relative to the total weight of the composition.

19. A process, wherein a composition as defined in claim 17 is applied to the skin, the hair, the eyelashes and/or the nails.

20. The process according to claim 19, wherein the composition is applied to sensitized keratin fibres.

21. The modified KAP polypeptide according to claim 1, wherein said polypeptide is isolated and/or purified and comprises at least one salified amino acid.

22. The modified KAP polypeptide according to claim 1, wherein said polypeptide is isolated and/or purified and comprises at least one amino acid modified by covalent bonding.
